(19) United States
(12) Patent Application Publication
(10) Pub. No.: US 2005/0177038 A1
(43) Pub. Date: Aug. 11, 2005

(54) Skin impedance matched biopotential electrode

(76) Inventors: Hans Kolpin, White Lake (CA); Izmail Batkin, Gloucester (CA); Riccardo Brun del Re, Ottawa (CA)

Correspondence Address: David J French, P O Box 2486, Station D, Ottawa, K1P 5W6, CA

(21) Appl. No.: 10/509,054

(22) PCT Filed: Mar. 26, 2003
(86) PCT No.: PCT/CA03/00426

(30) Foreign Application Priority Data
Mar. 26, 2002 (CA) 2,379,268

(51) Int. Cl. A61B 5/04

(52) U.S. Cl. 600/372; 600/395

(57) ABSTRACT

A bio-electrode for detecting heart signals and the like comprises a dry electode surface having an elevated resistivity to reduce the effect of polarization noise. The electrode is combined with a circuit having an external discharge resistor across which an output signal is obtained wherein the discharge resistor has a value which reduces the time constant of polarization noise to less than one second.

### FIELD OF THE INVENTION

This invention relates to the field of sensing voltage potentials arising from within a living body. More particularly, it relates to electrocardiogram-ECG electrodes for detecting heart signals and other body-originating potential signals such as for monitoring heart rate and cardiac pacemaker activity.

### BACKGROUND TO THE INVENTION

Electrodes for detecting electrical signals arising from within a living body may be classed, amongst other characteristics, as either wet- or dry-type electrodes. Wet-type electrodes operate on the basis of the presence of an electrolytic layer formed at the interface between electrode and the body surface that his provided as part of the electrode or as part of the standard electrode application process. Dry-type electrodes are intended to operate without the intentional addition of such an electrolytic layer but sometimes may require a natural layer of sweat or other fluids to function. It is noted that contemporary gel electrodes appear to present a gel surface which is dry. Nevertheless, such electrodes contain an electrolyte within the gel.

Electrodes may also be classified as being either ohmic or capacitive. Generally, ohmic electrodes are of the wet type, and capacitive electrodes are of the dry type.

All electrodes provide signals to associated circuitry by means of electron conduction, generally through metal wires. In ohmic electrodes of the wet type, materials that provide electron conduction are necessarily exposed to an electrolytic layer, typically in the form of an exposed surface that provides the interface between electrode and the body. Electron conduction arises with respect to metals, metal alloys, graphite, carbon black and other materials that display free-electron-type conduction with volume resistivity generally between 1 ohm-cm and 10⁻⁶ ohm-cm. Sweat formed on the surface of an electrode can serve as an electrolyte.

When a conductor is placed in contact with an electrolyte contact potentials are produced. A layer of ionic entities arise from within the electrolyte collects over the surface of such conductive material, providing what is known as Nernst polarization and otherwise being called the “half cell” effect. Similar polarization effects called “bilayers” arise whenever metals, and materials such as carbon which provide conduction based on electron flow, are immersed in a non-reactive electrolyte.

In a bio-electrode, the presence of a polarization effect gives rise to noise that interferes with the signal that is the focus of attention. Typical ECG bio-signals are of the order of one or two millivolts. Polarization noise arises when the ionic entities at the electrode interface are mechanically disturbed. Such noise is generally at 100 millivolt levels.

Changes in the sensor-to-body source resistance can lead to changes in signal levels at the reading device input and cause loss of common mode noise rejection efficiency.

Gel electrodes address these problems by striving to minimize resistance to body and by suppressing polarization noise by mechanically stabilizing this interface. Typically, in the case of gel electrodes the electrical signal is obtained through a conductor provided with a silver chloride surface layer that is immersed in an electrolytic gel containing chloride ions. This gel is held in contact with the skin of the patient generally by adhesive means rather than the traditional vacuum suction cups. In this manner mechanical disturbance of the surface over which the polarization entities are formed is minimized.

However, gel-electrodes are not reusable, have a limited shelf life and are uncomfortable for patients; they often cause skin irritation, particularly when worn for extended periods of time. Adhesives are a source of some skin irritation. Gel electrodes generally are not suitable to be worn for more than 72 hours. Gel electrodes may also produce a sizable direct current (DC) polarization voltage which requires additional interface circuitry to properly remove such off-sets from the desired alternating current (AC) signal.

It would be desirable to provide an electrode that does not require an aggressive adhesive attachment to the body nor rely upon provision of a gel that is susceptible to dehydration over time.

Polarization noise is not perceived to be a problem in capacitive electrodes. However, a highly insulative dielectric material is susceptible to the formation and/or presence of static electric charges at the electrode-body interface. These charges may arise in the form of local charge concentrations created within or upon the insulative stratum corneum layer of the skin or dielectric layer of the electrode through triboelectric effects. Since the dielectric material of a capacitive electrode is insulative, the presence of such material adjacent to such static charges, in the absence of a conductive electrolytic layer such as provided by sweat, does not contribute to the immediate discharging of such dipoles or charges. Consequently, mechanical disturbance of a capacitive electrode gives rise to noise artifacts associated with such static charges on dry skin. Noise from such static charges does not arise in the case of wet-type electrodes as the presence of the electrolyte layer and/or the conductive surface of the electrode minimizes the formation or persistence of localized potential differences at the electrode to body interface.

The high impedance of capacitive electrodes also makes them susceptible to radio-frequency, electromagnetic and other forms of electrical interference. Since capacitive electrodes have at least one conductive plate associated with them, such plates may act much like an antenna, picking up unwanted signals from outside the body.

It would be desirable to provide a reusable bio-electrode that need not necessarily be adhesively immobilized on the skin of the patient and need not necessarily rely upon a mechanically stabilized electrolyte-to-electrode boundary. At the same time, it would be highly desirable to minimize the noise effects arising from polarization and/or triboelectric phenomena.

As further background to the invention, it has been suggested in the literature that the polarization effect may be modeled, at the moment of the creation of a noise artifact, in respect of the circuit as it effects such noise artifact, as being equivalent to a capacitor momentarily present in the electrical circuit formed between the body and the electrode with its associated sensing components. For the purpose of this model in respect of its DC characteristics, a voltage source Vb is assumed to be present within the body, connected to the skin through:
a hypothetical resistance, largely dominated by the skin, and represented by a resistance, Rs;
the pseudo- or effective capacitance associated with the polarization, Cn; Cn is assumed to be momentarily present during a noise discharge. Otherwise it is treated as being absent, i.e. shorted.
a contact resistance Rc arising from imperfections in the electrode-to-skin contact;
the resistance arising from within the pickup electrode, Re;
the capacitance Ce formed across the pickup electrode, bridging Re;
the resistance across which the output signal is detected, typically dominated by a specific resistance bridged by the sensing circuitry but including the sensing circuitry input impedance as well, Ra;
the resistance of the return electrode connection to the body, together with its interface resistances, Rr, and
another resistance arising within the body, R's.
   Conveniently, the two body resistances, Rs, R's, may be consolidated for purposes of analysis into a single body resistance Rb. Further, as will be seen below, all resistances may be consolidated into a total resistance, Rt, of which the principal values of concern are Re and Ra.

These components govern the DC characteristics of the circuit. In fact, many of these resistive elements will display impedance characteristics that are frequency sensitive. Inductive aspects, parasitic or otherwise, are generally so small that their effects can be neglected. The capacitive effects are more significant, particularly in terms of signal capture ratios, but their presence does not derogate from the useful effects achieved by the invention. For the purposes of initial analysis, the following exposition will proceed on the basis of addressing DC or low-frequency behavior.

Collectively, the model circuit for polarization noise is equivalent at DC and low-frequency levels to the capacitor, Cn, being in series with the total of the listed resistances, wherein the combined capacitor and resistance elements have a time constant for the discharge of the capacitor characterized as the “RC” for this circuit. Here “R” corresponds to Rt for the entire circuit. This time constant, equal to RtCn, is the key parameter for determining the voltage Vc across the capacitor Cn as it discharges from an initial voltage of Vi, over time according to the exponential function exp (−t/RC). According to this function, the voltage Vc across the capacitor Cn will decline to 36 percent of its initial value in the period of one time constant RC; and to the only 0.6 percent of its initial voltage Vi in the period of five RC time constants.

The disturbance caused by a polarization noise artifact may therefore be characterized in one aspect by the time constant which is associated with this declining voltage effect. This is a fiction of the RC constant for the resistor-capacitor combination. The rate of decline of a voltage disturbance arising from a polarization effect, the “settling time”, should preferably be so rapid that it causes a minimum interference in the voltage waveform of the body event under examination.

Another issue relating to the detection of body potential signals is the extent to which the external sensing circuit can be provided with a voltage Va which corresponds to the signal Vb originating from the source within the living body. This may be referred to as the “signal capture ratio”.

Typically, all ECG systems rely on the formation of the closed circuit of elements as listed above with Cn assumed to be shorted. This circuit constitutes a voltage divider network. The output signal is obtained across the resistance Ra as referenced above. The signal capture ratio is provided by the usual formula:
% Ratio=Va/Vb=Ra/Rt
where Vb is the body source signal value, Va is the signal being measured across Ra, and Rt is the total resistance of the circuit. In cases where Ra and Re dominate as the largest resistances in the circuit, Rt reduces to Ra+Re.

Typical values for the area-resistivity of skin are 10⁴ ohm/cm² to 10⁶ ohm/cm² cf M. R. Prausnitz, Advanced Drug Delivery Reviews, 18 (1996) Elsevier Science p 395-425. For an electrode of total area 10 cm² this corresponds to representative skin resistance values in the range 10 exp 3 ohms to 10 exp 5 ohms. In cases of old, dry skin that is un-abraded, Rs can surpass 1 Mohm.

It has been taught in the past that the contact resistance Rc and skin resistance Rs should be minimized and that this percentage ratio Ra/Rt should be maintained at a maximum value in order to improve the signal to noise ratio in the output voltage Va being delivered to the amplifier. Accordingly, in past systems, efforts have been made to maximize the value of Ra with respect to the resistance values of other elements in the circuit, and particularly Re.

This object of maximizing the signal capture ratio, %, has been pursued in order to maximize the signal to noise ratio of the detected signal. However, a further consideration is to ensure that a gel-free electrode system will provide medical diagnostic quality outputs notwithstanding the high variability of electrode-to-skin contact resistances and/or impedances of patients. It would be an improvement in the art to provide a gel-free bio-electrode should preferably be able to perform satisfactorily on a large proportion of the population in circumstances where the electrode is being applied to unprepared skin. Some sacrifice in the capture ratio may be justified if this facilitates such other objectives.

Diagnostic quality performance has in the past been judged by the standard of obtaining signals in the range of 0.05 hertz to 100 hertz with signal noise levels not exceeding 20 microvolts, peak to peak. While not necessarily achieving this standard, the invention described hereafter will provide a satisfactory medical diagnostic level of signal that is substantially equivalent to the performance of typical gel electrodes.

Existing ECG systems generally rely on signal sensing and display circuitry having an input impedance of, on the order of 20 Mohm. In the case of heart rate pickups, generally utilized with sweat present, the input impedances of existing devices are usually lower than typical ECG device inputs, with heart rate device inputs possibly being on the order of 2 Mohms. However, the heart rate signal is normally derived principally from a sub-band of the diagnostic ECG signal—approximately 1 Hz to 20 Hz, and is therefore more tolerant of background noise. For this reason prior art “dry” electrodes have been sufficient for heart-rate pickup purposes on a majority of skin types.

Nevertheless, prior art heart rate electrode devices generally/typically fail to provide ECG quality signals on highly resistive skin due to the voltage divider constraint described above. The present invention represents an improvement over the prior art heart rate pickups by allowing ECG quality signal acquisition on skin of high resistance and by improving the signal to noise ratio.

One example of a prior art dry electrode systems is U.S. Pat. No. 4,122,843 issued Oct. 31, 1978 to Zdrojkowski (adopted herein by reference). In this reference a belt carries two pick up electrodes positioned against the skin to obtain body signals, and a third return electrode also held against the skin by the belt. The two pick up electrodes provide signals to a differential amplifier that minimizes common mode noise. In this reference the body-contacting electrode material is formed from a plastic loaded with electrically conductive particles, such as a mixture of silicone rubber or polyvinyl chloride and carbon particles. An amplifier input impedance of more than 10 Gohms is also proposed in this reference.

While the Zdrojkowski reference does not specify the resistivity of the electrode material, later attempts to build satisfactory dry electrodes include that described in the U.S. Pat. No. 4,865,039 issued Sep. 12, 1989 to Dunseath Jr. (adopted herein by reference). In this patent a resilient, dry electrode pad of low density, carbon-loaded polyurethane foam is provided, subject to the stipulation that this material should not establish an electrical impedance to the body of more than 1.5 million ohms at a frequency of 10 Hz.

According to another invention by Dunseath Jr outlined in U.S. Pat. No. 4,669,479 issued Jun. 2, 1987, (adopted herein by reference), a similar dry electrode is proposed, subject to the proviso that the bulk electrical resistivity of the material not be greater than 200,000 ohm-centimeters, and preferably between 800 ohm-centimeters and 3200 ohm-centimeters. This reference as well as other prior references, all reflect the assumption that it is desirable to minimize the resistance of the electrode at the electrode to body interface in order to maximize the signal capture ratio, thereby improving the signal-to-noise ratio.

It is against this background that the invention herein will now be presented.

The invention in its general form will first be described, and then its implementation in terms of specific embodiments will be detailed with reference to the drawings following hereafter. These embodiments are intended to demonstrate the principle of the invention, and the manner of its implementation. The invention in its broadest and more specific forms will then be further described, and defined, in each of the individual claims which conclude this Specification.

### SUMMARY OF THE INVENTION

The present invention relates to an improved type of dry electrode that can be used for pickup of signals from a living body.

This invention is based on the premise that it is advantageous in a bio-electrode to incorporate as the material for the body-directed face of the electrode a substrate material that has a lower level of conductivity than that commonly recommended. This selection of a higher resistivity material for the body-to-electrode interface is believed to reduce noise arising from polarization effects. According to one theory, this reduction occurs because a low conductivity substrate presents a smaller area of conductive particles forming part of the circuit within the electrode to be electrically connected to the body. This gives rise to a lower level of electrolytic contact noise. As a related consequence, the time constant for the discharge of noise artifacts arising from polarization effects can, in conjunction with the selection of appropriate external circuit elements, be reduced. This translates into reduced disturbances arising from noise.

By an alternate characterization, the invention is based on a bio-electrode produced from a material with sufficient bulk resistivity, as measured in a direction across the electrode (in a plane parallel to the body-facing surface) and within the layer providing the surface that is presented to the skin of the subject, to ensure that the material has a reduced tendency for polarization to form from within an electrolyte layer present at the electrode-to-body interface, thereby reducing noise voltages arising from disturbance of such electrolyte layer, e.g. polarization noise. At the same time, noise arising from static electricity is minimized by providing an upper limit to the resistivity of the material.

According to one aspect of the invention, a bio-electrode is provided that has, on the basis of a DC analysis and in respect of the electrode by itself, an electrode to body interface surface layer with a bulk resistivity ranging from 2×10 exp 5 to 10 exp 11 ohm-centimeters, as measured in a direction across (i.e. along) the body-directed face of the electrode at and just beneath the surface of the electrode that is presented to the skin of the subject. In conjunction with specific external circuit elements, such bulk resistivity can be as low as 10 exp 3 ohm-centimeters. More preferably, the bulk resistivity of the electrode at such interface, in the aforesaid direction, is in the range 10 exp 6 to 10 exp 10, even more preferably, in the range 10 exp 7 to 10 exp 10. Resistivity is preferably to be measured at low voltages, e.g. 10 volts per cm or less. This resistivity measurement may be made in any direction in a homogeneous material. Materials having graded levels of conductivity are preferably to be tested in the X-Y surface direction as specified above.

The objective of providing a bio-electrode with such a degree of resistivity is to reduce the extent to which polarization forms, arising from within an electrolyte layer present at the electrode-to-body interface, and therefore to reduce noise arising from polarization effects while maintaining enough electrical conductivity to allow low-level bio-signals to pass through and into the bio-electrode.

To achieve this objective, according to one variant of the invention, the bio-electrode has a body facing surface which comprises a plurality of relatively conductive areas or “islands” of conductivity, surrounded by portions of the body facing surface which are less conductive In this configuration, there is a depletion of conductive regions across the body-facing surface of the electrode and a corresponding reduction in electrolytic polarization. Preferably, the portions of the electrode surrounding the islands of conductivity are composed of a background material that does not associate strongly with polarizing entities. Such material should be relatively non-polarizable and nonconductive to avoid transmission of noise signals through the background material.

Enlarging further on this variant of the invention, the substrate of the body-facing surface comprises a non-conductive, background, supportive material rendered partially conductive by the addition of conductive additive that forms conductive pathways within the non-conductive, background material that extend to the requisite islands at the electrode-to-body interface. Conduction through the electrode may arise through “percolation” both above and below the percolation threshold, but preferably at conductivities below the percolation threshold. A suitable material for forming such extrinsic conductive pathways is carbon, preferably added in the form of carbon black, colloidal graphite or micro-fine carbon granules, embedded in a nonconductive support which serves as the background material.

According to an alternate variant of the invention, the electrode has a body-directed surface that is provided with a homogeneous layer of high resistivity biocompatible material which serves to establish a reduced population of polarizing entities over its interface surface area. It is believed that the high resistivity of the electrode substrate reduces the tendency for such polarizing entities to form at or remain in close proximity to the electrode-to-body interface.

Candidate materials for the background material are poorly conductive materials that have minimal chemical reactions with skin, sweat or skin lotions. Such materials should not generate significant internal electrical noise voltages such as those arising from strain-induced potentials, spontaneous polarizations (electret), contact polarizations or undue static electricity.

The material of the electrode may be based on rubber, plastic or glass that is otherwise sufficiently electrically inactive as to be compatible with achieving the objectives of the invention. To qualify as electrically inactive, the background material should have minimal or be substantially free of the following characteristics:
having internal electrical voltages
being an electret
being highly polarizable e.g. having a high dielectric constant
incorporating highly polarizable polymers
being chemically reactive with sweat, eg a ferrite
possessing acidic groups e.g. unreacted reagents in polymers.

The substrate background material should have low chemical reactivity, low intrinsic conductivity, low polarizability and low triboelectric (static) generation properties. Suitable materials include certain types of rubber materials, such as neoprene rubber, silicone rubber, nitrile rubber, butyl rubber, and numerous inert plastics. As indicated unsuitable materials include ferrites, ionic solids, dielectrics possessing electret properties or a high dielectric constant (polarizability) and air-cured silicones possessing acidic and/or polar groups.

By reducing the extent to which polarization binds charge within the electrode at the electrode-to-body interface, however this is made to occur, an opportunity is provided to reduce the impact on the output signal that would otherwise arise from polarization -generated noise. Modeling the source of this noise as being equivalent, at the moment of a noise discharge, to a capacitor present between the body and electrode at the body to electrode interface, it is a feature of the invention that this effective or “pseudo” capacitance is substantially reduced in its capacitive value. This effect allows use of an external signal-detecting circuit that:
1) provides for the rapid discharge of polarization-generated noise, and
2) still permits maintenance of a satisfactory signal capture ratio.

Thus, according to the invention, the electrode of the invention is combined with a signal sensing circuit wherein the total resistance and/or impedance in the closed circuit containing the source of polarization noise originating from the reduced-value pseudo capacitance of the polarization noise source is set to provide a time constant, RC, of a specific range of values. RC is established at a level that allows the polarization noise signal to be substantially discharged in a time period or “settling time” and that is minimally disruptive to the body signal.

Stated alternately, the time constant RC for the polarization noise signal should be reduced to less than one second, more preferably less than 100 milliseconds, even more preferably to less than 10 milliseconds.

These results may be achieved by selecting specific values for Ra and Re, including values limiting the distribution ratio for Ra/Re. In conjunction with the values for such resistances that make these two resistances the dominant resistances in the voltage divider circuit, this distribution ratio will become effectively the signal capture ratio. The preferred values for Ra range over 2 Mohms to 5 Gohms, more preferably 20 Mohms to 1 Gohm, still more preferably 100 Mohms to 1 Gohm. The ratio for Ra/Re may be in the range of over 1 to 1, more preferably over 5 to 1, and still more preferably 20 to 1 and higher.

As the closed circuit of the invention generally relies upon the presence of a second, return coupling to the body in order to close the circuit, a return electrode with a return electrode interface may generally be provided in association with the invention. When employed as the common return for a dual mode, differential noise reduction circuit, the return electrode Rr may be of a conventional low resistivity type. Polarization noise arising at this interface will consequently become cancelled by common mode noise rejection.

up with a with both of a fifth A dual-pickup, common noise rejection canceling circuit is based upon the differential comparison of two separately detected body signals. To be fully effective, a common mode noise rejection circuit should have balanced input connections on each input channel. By employing high Re and Ra values, the imbalancing effects of variable skin Rs and contact Rc resistances are reduced. Accordingly, it is a preferred embodiment of the invention that two pickup electrodes, each incorporating an electrode interface as stipulated above, provide signals to a differential amplifier that has a grounded return coupled to the body and provides an output signal that has been obtained from the two pickup electrodes with common mode noise rejection.

Due to the fact that noise can arise through the leads coupling the electrode to a signal display apparatus (“whip” noise), it is preferable that the electrode be an “active” electrode that is provided with high input impedance circuitry, approximately located at the electrode, and which may serve to provide a low output impedance to the cables extending to the display apparatus. Ideally, this circuitry can be in the form of on-board electrical components that are supported within the same structure as the electrode. Such “onboard” circuitry provides a high input impedance buffer circuit which serves as an impedance converter. Power for this circuitry can be supplied in DC format through the same connecting cable that delivers to the display apparatus the signal that corresponds to the actual sensed signal. Alternately, an internal battery or other types of power sources can provide power.

Conveniently, shielding can enclose not only the cables but also the circuitry to minimize interference arising from ambient electromagnetic or radio-frequency noise signals. Thus the invention may incorporate a shield overlying the electrode, said shield being:
(1) provided with an insulating gap to prevent its contact with the electrode substrate;
(2) coated or embedded in a insulating and waterproofing material; and
(3) electrically connected to the reference potential for the electronic circuit used to convey the detected signal to the display apparatus.
   This circuit may beneficially be shielded in a manner similar to those described in U.S. Pat. No. 6,327486 issued Dec. 4, 2001 (adopted herein by reference).

The foregoing summarizes the principal features of the invention and some of its optional aspects. The invention may be further understood by the description of the preferred embodiments, in conjunction with the drawings, which now follow.

### BRIEF DESCRIPTION OF THE DRAWINGS

FIG. 1 is a pictorial schematic of an electrode according to the invention presented to the body of a subject, together with associated external electronic circuitry, before taking into consideration polarization noise effects.

FIG. 2 is a variant electrical schematic to that of FIG. 1 wherein a noise source capacitor Cn is momentarily present, modeling polarization noise effects.

FIG. 3 is a cross-sectional side view of an active electrode made in accordance with the principles of the invention.

FIG. 4 is a graph of the time constant Tau for a hypothetical polarization noise source capacitance Cn as in FIG. 2 as a function of the bulk resistivity Rho for the surface layer of an electrode according to the invention.

FIG. 5 is variant graph on the graph of FIG. 3 wherein Cn is assumed to have a minimum value of 30 picofarads based on tribo-electrical noise generated at the electrode-to-body interface.

FIG. 6 shows two simultaneous ECG traces obtained on a patient, the upper one based on a standard event recorder using gel electrodes, the other lower trace showing the same events as recorded by electrodes according to the invention.

FIG. 7 shows the frequency band pass characteristics of a circuit incorporating electrodes according to the invention.

FIG. 8 is a systematic for a differential electronic circuit that operates to minimize common mode noise.

FIG. 9 is a schematic depiction of a hypothetical, enlarged cross-section of the substrate of an electrode according to the invention depicting hypothetical capacitors and resistors that may make up such substrate.

### DESCRIPTION OF THE PREFERRED EMBODIMENT

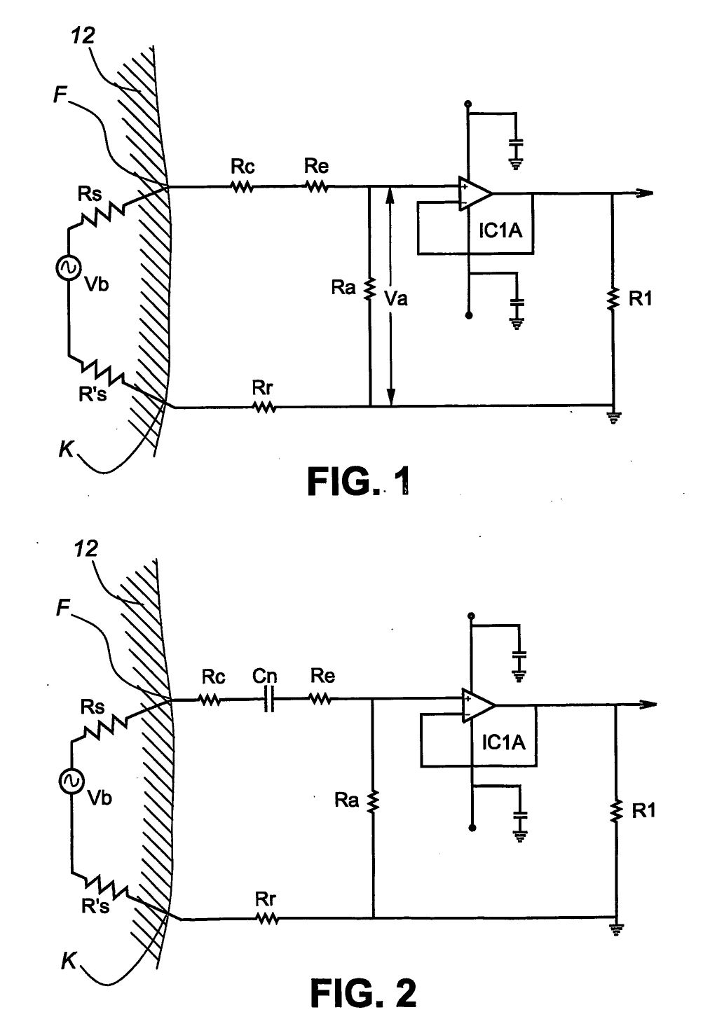

FIG. 1 depicts a pictorial schematic layout for the electrical circuit of the invention, when analyzed in terms of DC currents, before taking into consideration polarization noise effects.

All pickup electrodes are used to convey signals originating inside a body 12 to an external reading device such as an ECG machine or heart rate counter through a closed circuit which provides a voltage divider network. The electrical signal inside the body can be called the body-source, as represented by a voltage Vb. Analyzing this circuit for its DC characteristics, the body source, along with the voltage divider required for the pickup of the bio-signal is illustrated in FIG. 1 wherein:
Rs and R's are the skin resistance;
F is the location of the body-to-electrode interface;
Rc is the contact resistance at the interface F;
Re is the electrode bulk resistance, and
Ra is the resistance across which the output signal Va is measured.

The end of the voltage divider, opposite to the electrode, is connected to the body through Rr at point K. Though showing as a resistor, Rr in particular may also be provided as an impedance having a significant capacitance component to reduce its impedance in the frequency band of interest. This closes the circuit to provide the voltage divider network. An operational amplifier, ICIA, serves as the sensing electronics.

The total resistance Rt of the circuit is approximately given by the sum of the sensing resistor Ra, the bulk electrode resistance Re, the skin resistances Rs, R's plus the contact resistance Rc arising at the electrode-to-skin interface. The contact resistance at the return electrode location K is assumed to be minimal because the return electrode preferably establishes a very high conductivity connection to the body.

In the case of passive electrodes connected to an ECG machine, Ra represents the ECG machine input resistance. In the case of active, ohmic pickup electrodes possessing an on-board, internal buffer amplifier acting as an impedance converter, Ra represents the combined resistance of the sensing circuit as bridged by the sensing resistor.

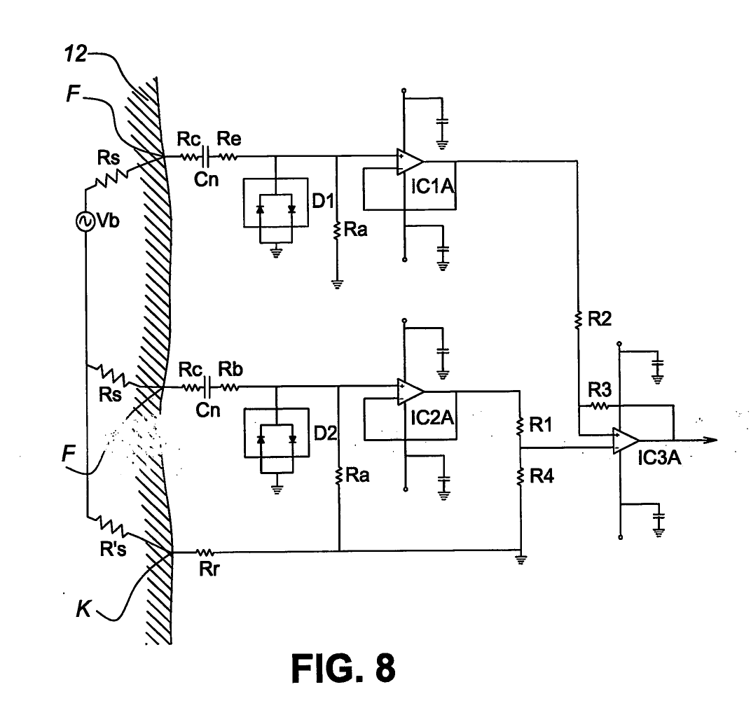

In order to protect the sensing circuitry from overload voltages, Ra may be paralleled by two parallel, reversely oriented diodes such as Schottky, low leakage diodes exemplified by Panasonic MA198CT. Diodes D1, D2 are shown in FIG. 8. At the low signal levels provided by the pick-up electrodes, such diodes exhibit high forward resistances, having a resistance of on the order of 10 exp 12-13 ohms. The forward resistance of Schottky diodes before breakdown occurs is at on the order of 10 exp 13 ohms. By choosing diodes with a forward breakdown voltage that is above the level of the signal of interest, the “reset” function of the input resistance of the high impedance amplifier can be improved.

It is often recommended for bio-signal pickup including ECG that skin preparation such as cleaning, shaving and abrasion be performed to ensure that the skin resistance (Rs) attains the lowest possible value. The present invention represents a departure from the prior art by providing an electrode that does not require substantial skin preparation to produce high quality signals. However, naturally forming sweat can improve performance and this effect can be accelerated by providing moisture at the electrode-to-body interfaces, F, K.

In FIG. 2 the noise generating aspect of polarization is modelled as a capacitor Cn which may be envisioned as having been charged by a battery with fixed DC voltage, Vn, which capacitor Cn is randomly switched into and out of series connection with Re. Polarization noise arises when Cn randomly discharges into the voltage divider.

The value of Ra may be chosen by the requirement that the measured output signal Va should be at least generally half that of the body source voltage Vb or preferably larger. For example, if it is desired that Va should be in magnitude 95% of Vb, then Ra should be about 20 times the value of Re. It is permissible, however, for Ra to be less than Re, but at the expense of a reduced output signal Va.

When Ra and Re together are much greater than Rs etc, the electrode output signal Va is approximately governed by the signal distribution relationship:
Va=Vb[Ra/(Re+Ra)]
where Vb is the body voltage and Va is the sensed voltage (across Ra).

For reasons analogous to those discussed above in connection with impedances of typical reading devices, the resistor Ra should not be much larger than that required to satisfy signal size requirements because overly large values for Ra can introduce noise or compromise the desired signal-stabilizing and referencing properties of the invention.

The return electrode Rr contact at point K is not shown in FIG. 2 as being a source of noise for simplification. The return electrode preferably makes a very high conductance contact with the body. By utilizing dual pick-up electrodes to effect common mode noise rejection, noise effects arising at the contact K can be ignored. The reference electrode at point K can preferably take the form of a low resistance, passive, dry (or wet) electrode of standard ohmic type so long as it is used in combination with a differential noise rejection circuit. Alternately, it can simply be an electrode according to the invention, but with minimal resistivity.

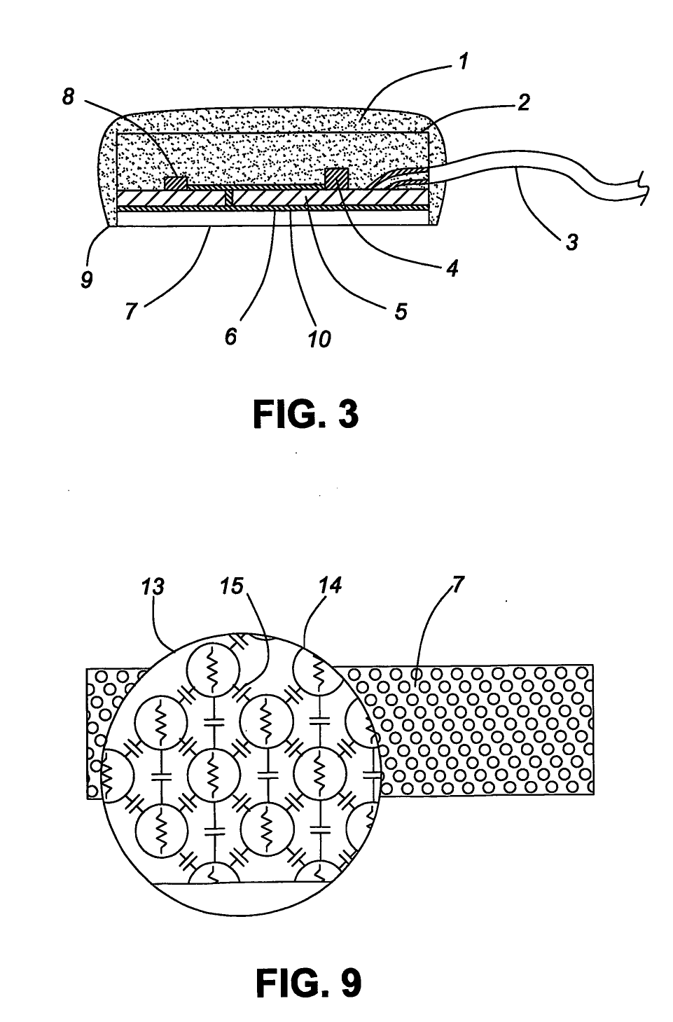

FIG. 3 illustrates a cross-sectional view of a coin-shaped or disc-shaped electrode of the invention. The electrode is encapsulated with an insulating layer 1 which is electrically resistive and waterproof. Several encapsulating materials including non-conductive epoxy, plastic and rubber compounds have been found suitable for this purpose. The electrode possesses an internal conductive cap acting as a shield 2, which is “grounded” i.e. connected to the circuit reference potential which is connected to the reference electrode. A cable 3 carries power to, and signals from the on-board electronic circuit 4. The circuit 4 is fixed on a 2-layer printed circuit board 5 with a bottom conducting layer 6 conveniently serving as the low resistance ohmic contact to the electrode substrate layer 7. That substrate layer 7 is about 6 cm\hat{ }2 in area.

A preferred material for substrate layer 7 is a moulded-rubber sheet containing a suspension of fine carbon to render it mildly conducting according to the invention. Various mixtures with desirable resistivities can be made in accordance with the teachings of “Conductive Rubber and Plastics”, R. N. Norma, Elsevier Publishing Co. Amsterdam 1970. Successful electrodes have been constructed using olefin elastomers including EPDM (Ethylene Propylene Diene Monomer), neoprene and butyl-, nitrile, and silicone-based rubbers that are rendered slightly conductive with carbon black, or with other conductive additives that form a conductive matrix in the background, non-conductive, support material. The invention however relates to any substrate materials possessing low bulk conductivity of the desired value as well as the other appropriate characteristics.

The substrate layer 7 may be bonded to the conducting layer 6 by way of a conductive adhesive. Alternately, substrate layer 7 can be painted or moulded onto the circuit board conducting layer 6. The substrate layer 7 may have a volume resistivity in the X-Y plane of the electrode surface in the range 10 exp 3 ohm-cm to 10 exp 11 ohm-cm, which is a primary range for the invention. The resistivity characteristics of the invention are stipulated as being measured in the plane of the electrode surface because polarization arises on this surface. The Re value of this electrode of FIG. 3 was approximately 1 Mohms impedance and was employed with an Ra of approximately 1 Gohm.

The insulating layer 1 may extend to a point along the outer edges of the electrode so as to present an insulating ring around the substrate on the body-facing side of the electrode. A grounding ring (not shown), connected to the circuit ground, may surround the insulating ring, positioned to contact the body and provide a supplementary or alternate primary ground.

Electrodes of the invention have the advantage of producing very low ½-cell or polarization noise. This is believed to be due to the poor conductivity of the substrate on the following basis. This basis is presented as a theory that need not necessarily be correct.

An electrode based upon a conductive additive distributed within an insulative background material can be envisioned as a parallel array of many microscopic electrodes seen as series elements extending from the body-facing side of the substrate to the sensor input. Each element can be considered to terminate on a small capacitor C*n, representing the ½-cell capacitance due to the contact between the small element and the body. Each electrode element also comprises a resistor R*e representing the resistance of the overlying substrate layer responsible for conducting the bio-signal into the sensor. The complete electrode is an inter- or cross-connected parallel network of such elements with combined capacitance Cn equal to the sum of all the C*n and combined resistance Re arising from the interconnected sum of all the R*e.

An electrode of the invention with high resistivity (low conductivity) can be considered to be a microscopic network of a few interconnected, parallel electrode circuits suspended in a non-conducting background material. At the electrode surface, the conductive links terminate at small islands, surrounded by the background material. The elemental capacitors C*n that are responsible for polarization noise are located at these small islands. Since the total polarization capacitance generated by the electrode is the sum of the elemental capacitances, a substrate with high resistivity (low conductivity) produces a lesser total Cn than an electrode of substrate with low resistivity.

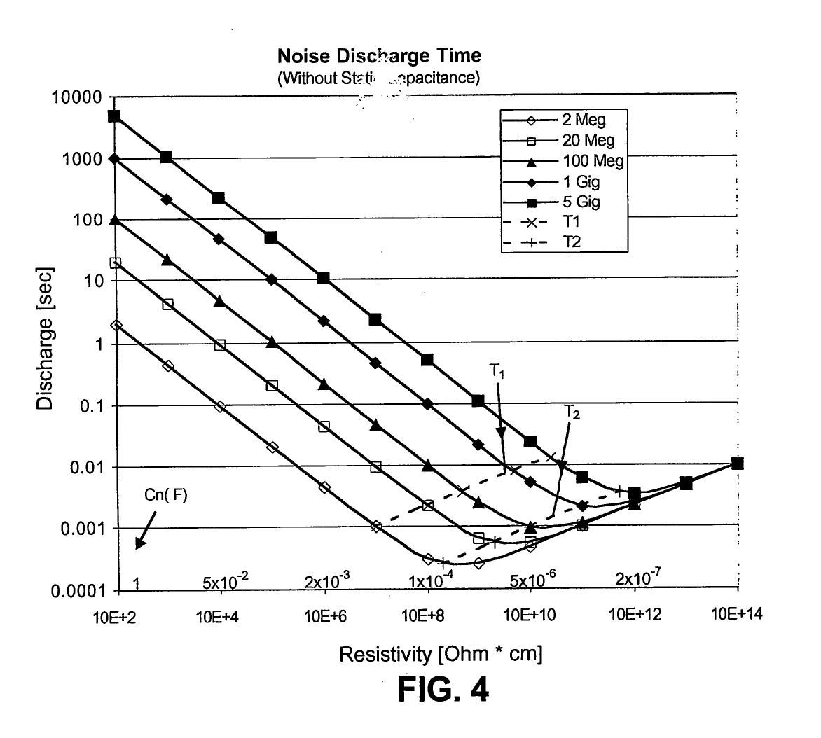

Using the electrical schematic of FIG. 2, FIG. 4 sets forth a graph which is intended to demonstrate the principle of the invention. While based upon certain hypothetical assumptions, FIG. 4 indicates how the time constant for polarization noise, Tau, can be reduced by employing increasingly larger volume resistivity values, Rho, for the body-facing surface 10 of the pickup electrode.

Thus FIG. 4 is a graph of a hypothetical time constant ordinate, Tau, wherein Tau most accurately equals CnRt. However, for simplification this graph has been prepared using the formula Tau=Cn (Re+Ra). This approximation becomes accurate when Rt essentially equals Re+Ra.

This time constant Tau assumes an electrode substrate in the form of a 10 cm square plate area and a 1 mm. The abscissa plots volume resistivity, Rho, for the layer of the electrode occupying the gap between the electrical circuit side conductive plate 6 of the electrode and the body side of the electrode. Both Tau and Rho are plotted on logarithmic scales.

Re is assumed to be proportional to the bulk resistivity Rho (Re equals Rho×thickness/area). Cn is assumed to be proportional to 1/Rho exp 2/3. This is based on the assumption that surface area varies as a two-thirds power of volume. The capacitance Cn is presumed to be proportional to the portion of the surface area occupied by islands of conductivity connected to conductive pathways through the electrode.

In the case of conductive particles randomly suspended in a volume of insulating medium, it is known that the surface density of conductive particles is proportional to the volume particle density raised to the power 2/3. The conductivity of such a medium is a highly non-linear function of the particle concentration. In this case, the conductivity (1/Rho) as a function of particle concentration undergoes a sharp increase at a specific conductive additive concentration called the percolation threshold (Pc). At lower concentrations, very few of the conductive particles participate in conductivity through the layer because many occur in isolation, with no significant electrical contact to neighboring particles.

In the model: Cn varies as Rho exp −2/3, we have assumed that the number of networked conductive particles is proportional to the DC volume conductivity (1/Rho) and that the effective, conductive area of the electrode is proportional to the number of networked conductive particles that occur on the surface i.e. proportional to the number of networked particles raised to the power 2/3. This results in Cn proportional to (1/Rho exp 2/3).

On this basis, FIG. 4 is plotted for demonstration purposes on the initial premise that Cn has a value of 1 microfarad for a Rho value of 100 ohm-cm. Various curves for Tau are shown corresponding to fixed values for Ra, e.g. 2, 20, 200 Mohms and 1 Gohm. Ra should generally exceed Re and preferably be as high as 20 times Re, e.g. a signal distribution ratio between Ra and Re of approximately 95 percent. But achieving such a high capture ratio is not essential.

Different values for Ra are relevant as the formula Tau=Cn×Rt only reduces to Tau=Cn (Re+Ra) when Re and Ra dominate all other resistances in the circuit. The other resistances include body resistance Rb, contact resistance Rc (which is highly variable and may typically range on the order of 50 kohms to 5 Mohms per cm²), and return electrode resistance Rr. The total of such resistances will typically not exceed 3 Mohms, or more certainly, 5 Mohms, for a large majority of persons. For simplification, Rt is assumed to be equal to (Re+Ra) in plotting FIG. 4. For high values of Ra and Re, the signal distribution ratio Ra/(Re+Ra) is essentially the signal capture ratio.

While unprepared skin resistance is typically estimated at 300 kohms per cm², it can range below 100 kohms/cm², and up to about 2 Mohms/cm². Accordingly, the curve for Ra=2 Mohms does not meet the assumption that Rt is essentially equal to (Re+Ra). However, for Ra=20 Mohms, this equivalence is more nearly true. And even more so for Ra=100 Mohms and higher.

Nevertheless, some degree of useful performance of the invention can still be obtained in some cases where Ra values are as low as 2 Mohms, subject to the difficulty that common mode noise rejection may not be as effective for such low values of Ra. On the other hand, it is preferable that the value for Ra not exceed a 10 Gohm value, more preferably not exceed 5 Gohms, and even more preferably, be less than one Gohm. This is to avoid the introduction of noise artifacts arising from static charges.

The curves all descend while the value for Cn falls as Rho increases. Cn dominates the term Ra+Re while Re is less than Ra. But when Re becomes larger than Ra, the curve for Tau changes towards increasing values of Tau with increasing values of Rho. This curve for Tau thereafter increases in FIG. 4 at a rate proportional to Rho exp 1/3. The “knee” in the curve identifies the shift from Ra being predominant over Re to the stage where Re predominates over Ra.

Shown on both FIGS. 3 and 4 is a trace Ti in the form of a line indicating the boundary where Ra=20 Re. To the left of this trace, Ra is greater than 20 Re. To the right this distribution ratio drops below 20 to 1. A second line T2 traces values for Ra=Re. For preferred high capture ratios, electrodes of the invention should be designed to operate to the left of these traces.

As it is desirable to avoid variable performance arising from variations in the skin Rs and contact Rc resistances, it is also preferable to operate with Ra values above on the order of 2 Mohms, more preferably above 20 Mohms and even more preferably above 200 Mohms.

As the object is to reduce the effect of polarization noise arising from Cn, electrodes according to the invention should preferably have a Tau of less than one second. More preferably the Tau should be less than 100 milliseconds and even more preferably 10 milliseconds or less.

To complete the definition of the preferred operating regime of the invention, it is believed that values for Rho in excess of 10 exponent 11 ohm-cm should be avoided due to the increasing noise effects arising from slow discharge of static/tribo-electric charges, such as may develop across dry skin.

The upper limit of the regime of substrate resistivity of the invention, i.e. 10 exp 11, more preferably 10 exp 10 ohm-cm is believed to define the practical limit for the realization of the advantages of the invention. This is because the advantages of the high resistivity substrate, namely the reduction of polarization effects, are countered by the onset of a secondary noise generation mechanism i.e. triboelectricity, also called static electricity, that is formed by contact between the virtually insulating electrode substrate and the body. As the substrate resistivity Rho increases above the order of magnitude 10 exp 10 ohm-cm and the corresponding Ra increases, the reduction in the polarization effect increasingly becomes counter-balanced by the increasing significance of triboelectric charges and surface charge effects which create noise voltages.

Concurrent increases in Ra creates a situation whereby the discharge times for these noise sources also increases. In fact, electrodes with substrate resistivity substantially above the order 10 exp 10 ohm-cm begin to operate akin to a capacitive mode. Thus it can be said that electrodes of the invention, particularly for the purpose of ECG measurements, operate in a “crossover” regime between ohmic and capacitive operation.

It has been found that experiments with electrodes of low-capacitance type as specified in PCT application PCT/CA00/00981 (adopted herein by reference) that fully capacitive operation is realized with substrate resistivities greater than 10 exp 14 ohm-cm and input bridging Ra values of the order 10 exp 12 ohms. In these ranges in PCT application PCT/CA00/00981 Ra is preferably limited to provide for the discharge of the electrode capacitance when disturbed by noise signals occurring below the frequency band of interest e.g. below 0.05 Hz.

It will be seen from FIG. 4 that a preferred region for the operation of an electrode according to the invention is in the lower portion of the defined area of the graph wherein:
1) Tau is minimal;
2) the distribution (and capture) ratio is higher;
3) Ra is sufficiently large so as to desensitize the electrode from variations in skin and contact resistances, but not so large as to make the system sensitive to static charge and tribo-electric effects or environmental interference; and
4) Re is sufficiently large so as to achieve the above trade-offs, namely: provide a reduced value for Tau, (thereby desensitizing the electrode to noise arising from polarization effects) but not so large as to extend the time period for the discharge of noise from static charge and tribo-electric effects or reduce the capture ratio below 1 to 1.

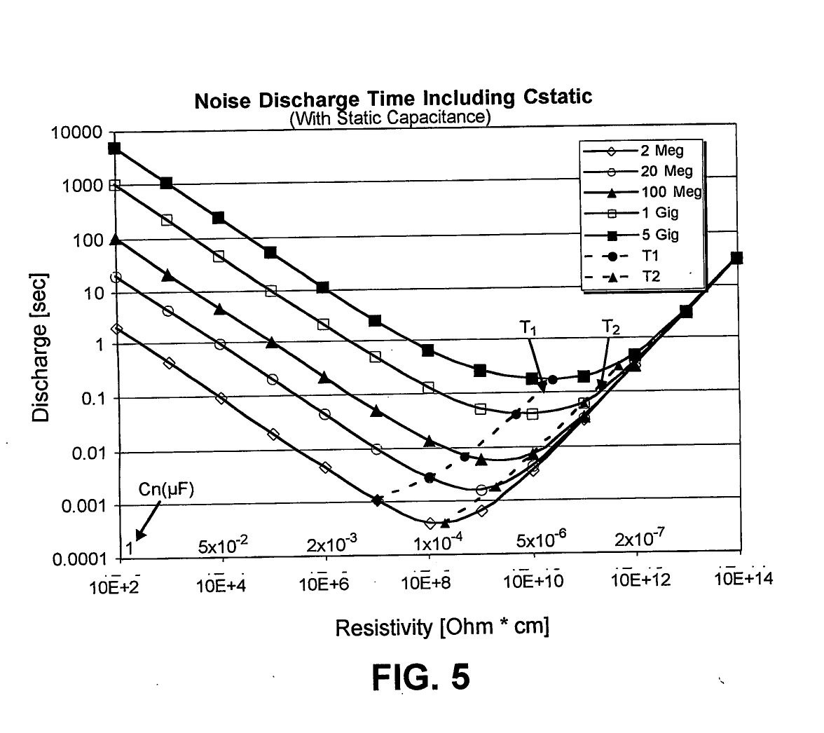

FIG. 5 shows a variation over FIG. 4 wherein a background fixed capacitance of 30 picofarads is assumed to be present in addition to Cn. This assumption allows for the presence of residual, intrinsic capacitance at the electrode-to-body interface that arises from overall geometry considerations and may hold static charge.

In FIG. 5, to the right of the “knee”, the curves for Tau increase more rapidly than in FIG. 4. Transverse traces for distribution ratios of 20 to 1 (T1), and 1-to-1 (T2) are shown on both FIGS. 4 and 5, indicating that the preferred region for operation of electrodes of the invention is not significantly modified by the assumption that Cn reaches a minimum, constant value of 30 picofarads.

In terms of the preferred operating region of the invention, as previously defined, it will be noted that Dunseath Jr., in U.S. Pat. No. 4,669,479, recommended use of an electrode material with a Rho not exceeding 2×10 exp 5 ohm-cm and an Ra of greater than 10 Gohms. In terms of the relevant surface layer of the electrode, the inventors do not claim electrodes by themselves having a Rho of less than 2×10 exp 5 ohm-cm. However, in combination with the range of preferred values for Ra, the invention may operate with Rho values of less than 2×10 exp 5 ohm-cm. It is believed that the invention will work with Rho values commencing from about 10 exp 3 ohm-cm and higher in conjunction with an electric circuit having the preferred values for the various components as outlined above.

While the invention has been described in terms of the DC characteristics of the electrode and sensing resistor Ra, many of the elements of the circuit may qualify as impedances wherein the reactive component of the impedances arises from capacitive effects. A principal circuit component in this regard is Re. Re in one simplified interpretation may be considered to be bridged by a single parallel bulk capacitance Ce. In a more elaborate analysis the electrode substrate may be modeled as depicted in FIG. 9. The actual capacitive character of the high resistivity substrate of the invention has been tested and found to be highly complex. Capacitive value measurements have been found to be frequency dependant.

FIG. 9 addresses a possible explanation for the source of the complexity of the impedance characteristics of an electrode made in accordance with the invention. In the simplest view of a carbon-loaded rubber 13, the particles 14 each have resistance, and the space between the carbon particles 14 has a certain capacitance 15. This is depicted in FIG. 9. In addition there will be some chains of particles which have purely DC resistance (not shown).

The capacitors 15 are significant in value because capacitance is inversely proportional to the insulating gap size. Since these particles 14 are very close together, their capacitance is large. The capacitors 15 are in a mass of series and parallel configurations, but when taken in totality provide a specific, and possibly frequency dependent, bulk-capacitive component for Ce.

Such complexity does not, however, affect the time constant Tau, arising from Cn. Rather, it may affect the capture ratio. In fact, significant values for Ce will increase the capture ratio for higher frequency signals.

In these circumstances it is believed that the DC analysis of the circuits as provided fairly characterizes the presence of the invention.

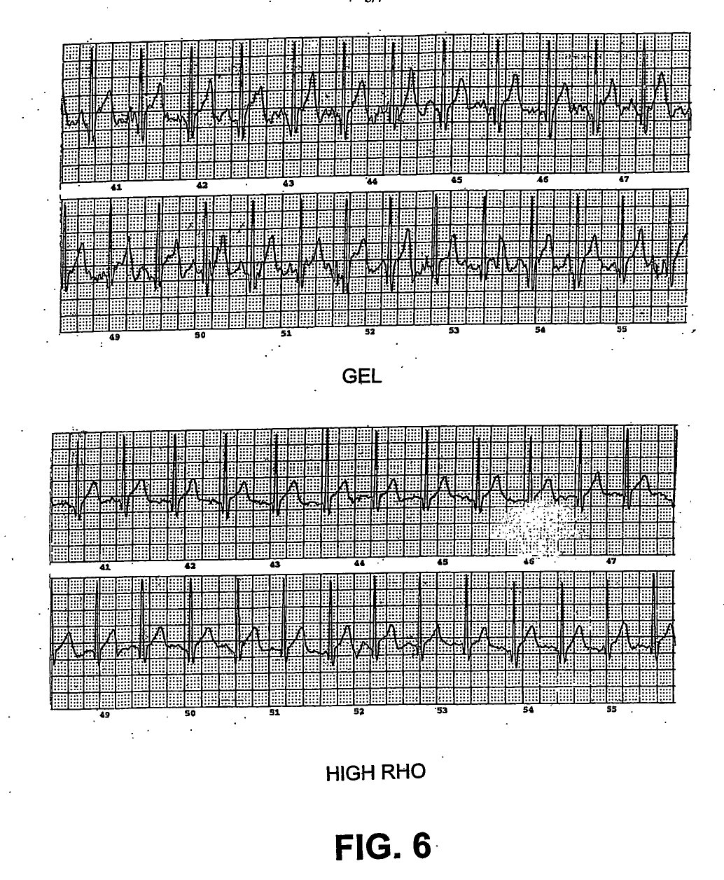

FIG. 6 shows simultaneous signals comparing standard gel electrodes with two active electrodes according to the invention. The different sets of electrodes were applied to skin of a patient at adjacent locations on the chest just beneath each breast. The gel electrode sites were prepared according to standard protocols for ECG procedures (top trace). Such electrodes were applied to cleaned, abraded skin of the patient and subsequently connected to one of the identical event recorders. The upper trace shows the signal derived from the two passive medical adhesive gel electrodes.

The lower trace shows the signal obtained by connecting the second of the identical event recorders to two active electrodes of the type illustrated in FIG. 2. The electrodes of the invention were moistened with a damp sponge and applied to adjacent unprepared skin of the same patient.

Each trace was recorded using the same type of single-channel output commercially available event recorder connected through two-lead wire cables to a pair of electrodes. The output signal in both cases was based on common mode noise rejection. During the time of the recording in each case, the patient was in a state of motion.

The signal quality is significantly higher in the case of the electrodes of the invention in that less noise is present.

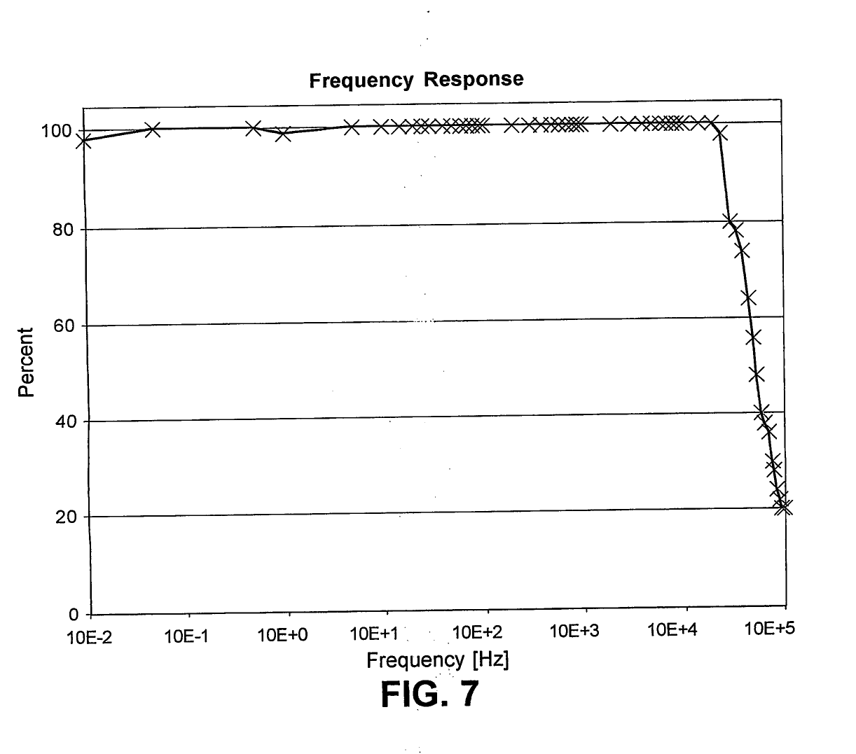

FIG. 7 depicts the band pass characteristics for an electrode module built based on the design of FIGS. 2 and 8. FIG. 7 shows that signals applied to the electrode are delivered by the sensing circuitry with a virtually flat band pass response over the range from 0.01 Hertz to over 20 kilohertz.

FIG. 8 shows a differential input electronic circuit that reduces or eliminates common mode noise. In FIG. 8 two pick-ups similar to that of FIG. 2 are used to drive a differential amplifier pair IC1A, IC2A. The additional operational amplifier IC3A further conditions the signal for transmission by shielded wire 3 to a display or recording device.

By use of this differential signal detection circuit, common mode noise arising from the return electrode connection will be minimized.

### CONCLUSION

The foregoing has constituted a description of specific embodiments showing how the invention may be applied and put into use. These embodiments are only exemplary. The invention in its broadest, and more specific aspects, is further described and defined in the claims which now follow.

These claims, and the language used therein, are to be understood in terms of the variants of the invention which have been described. They are not to be restricted to such variants, but are to be read as covering the full scope of the invention as is implicit within the invention and the disclosure that has been provided herein.

1. A Bio-electrode with a biocompatible electrode-to-body interface surface layer providing a body-directed surface, said surface layer having on the basis of a dc analysis a bulk resistivity, as measured in a direction across said surface layer, parallel to the plane of said surface, ranging from 2×10 exp 5 to 10 exp 11 ohm-centimeters and having a reduced tendency for polarization to be formed within an electrolyte layer when present at the electrode-to-body interface and thereby reducing polarization noise.

2. A bio-electrode with a biocompatible electrode-to-body interface surface layer providing a body-directed surface, said surface layer having on the basis of a DC analysis a bulk resistivity ranging from 10 exp 3 to 10 exp 11 ohm-centimeters, as measured in a direction across said surface layer, parallel to the plane of said surface, and having a reduced tendency for polarization to be formed within an electrolyte layer when present at the electrode-to-body interface and thereby reduce polarization noise,
in combination with external circuit components for providing a closed circuit with a closed circuit path extending through the body, the bio-electrode, and the external circuit components, said circuit comprising the following features:
a) said bio-electrode having an electrode resistance value of Re,
b) the circuit including an amplifier resistive element which, together with a high impedance signal sensing circuit connected across such resistive component, has a composite resistance value of Ra, and
c) Ra and Re being in series
wherein the value for Ra is between 2 Mohm and 5 Gohm.

3. A bio-electrode as in claim 1 wherein said body-directed surface comprises a plurality of relatively conductive areas or “islands” of conductivity connected to conductive pathways passing through the bio-electrode, said islands constituting a reduced portion of the surface area of the body-directed surface and being surrounded by portions of the body-directed surface provided by a generally nonconducting background material of the electrode, which portions have a reduced affinity to attract polarization from within an electrolytic layer when present at said surface sufficient to provide a reduction in the total polarization that will form across the body-directed surface of the electrode.

4. A bio-electrode as in claim 1 wherein the electrode has a substrate providing the surface layer which substrate comprises said background material, and wherein said background material is rendered partially conductive by the presence of conductive additive that forms said conductive pathways within the background material.

5. A bio-electrode as in claim 3 wherein the background material of the electrode is a material that is relatively non-polarizable and has a bulk of resistivity of in excess of 10 exp 12 ohm-cm.

6. A bio-electrode as in claim 3 wherein the background material is the composed of a material selected from the group consisting of neoprene rubber, silicone rubber, nitrile rubber, butyl rubber, EPDM, and olefin elastomers.

7. A bio-electrode as in claim 3 wherein the conductive pathways provide conduction through the electrode by means of “percolation”.

8. A bio-electrode as in claim 7 wherein such conductive pathways comprise carbon.

9. A bio-electrode as in claim 8 in wherein such conductive pathways are constituted by carbon.

10. A bio-electrode as in any one of claims 2 to 9 wherein said circuit comprises:
a) a total resistance R in the closed circuit wherein R equals (Re+Ra+3 Mohms), and
b) a source of polarization noise wherein the source of polarization noise is equivalent, at the moment of a noise discharge, to an effective or pseudo capacitor C present between the body and electrode at the body to electrode interface, and
wherein the values of R and C provide a time constant, RC, of one second or less for the polarization noise.

11. A bio-electrode as in claim 10 wherein the time constant RC for the polarization noise signal is less than 100 milliseconds.

12. A bio-electrode as in claim 10 wherein the time constant RC for the polarization noise signal is less than 10 milliseconds.

13. A bio-electrode as in claim 1 wherein said bulk resistivity is in the range 10 exp 6 to 10 exp 10.

14. A bio-electrode as in claim 1 wherein said bulk resistivity is in the range 10 exp 7 to 10 exp 10.

15. A bio-electrode as in claim 2 wherein the ratio for Ra/Re has a value of 1 to 1 or higher.

16. A bio-electrode as in claim 15 wherein the ratio for Ra/Re has a value of 5 to 1 or higher.

17. A bio-electrode as in claim 15 wherein the ratio for Ra/Re has a value of 20 to 1 or higher.

18. A bio-electrode as in claim 2 wherein the value for Ra is between 20 Mohms and 5 Gohms.

19. A bio-electrode as in claim 18 wherein the value for Ra is between 100 Mohms and 5 Gohms.

20. A bio-electrode as in claim 18 wherein the value for Ra is between 200 Mohms and 1 Gohms.

21. A bio-electrode as in claim 1 wherein the body-directed surface of the electrode is substantially non-adhesive.

22. A bio-electrode as in claim 2 wherein the circuit components provide a minimum band pass range of from 0.05 hertz to 100 hertz for signals originating from a body for the measurement of ECG signals.

23. A bio-electrode as in claim 22 wherein the circuit provides a band pass of from 1 hertz to 100 hertz for the measurement of heart rate signals originating from a body.

24. A bio-electrode as in claim 2 wherein the circuit provides a band pass of from 1 kilohertz to 20 kilohertz for the monitoring of pacemakers present within a body.

25. Two bio-electrodes as in claim 1 in combination with a common mode noise differential sensing circuit whereby common mode noise presented to the differential sensing circuit by the respective bio-electrodes is canceled.
